(19) United States
(12) Patent Application Publication
(10) Pub. No.: US 2025/0061987 A1
(43) Pub. Date: Feb. 20, 2025

(54) SYSTEM THAT INTEGRATES GENOMIC DATA AND ELECTRONIC HEALTH RECORD (EHR) DATA IN DEFINING A COHORT

(71) Applicant: Helix, Inc., San Mateo, CA (US)

(72) Inventors: Evanette Burrows, Loxahatchee, FL (US); Lisa McEwen, Victoria (CA); Francisco Tanudjaja, Redwood City, CA (US); Basil Khuder, Houston, TX (US); Simon White, Redwood City, CA (US); Olivia Kezirian, Phoenix, AZ (US); Kent Shefchek, New Market, MD (US); Nicole Washington, Albany, CA (US); Alexandre Bolze, San Francisco, CA (US)

(21) Appl. No.: 18/235,250

(22) Filed: Aug. 17, 2023

(51) Int. Cl. G16H 10/60; G16H 15/00

(52) U.S. Cl. CPC G16H 10/60; G16H 15/00

(57) ABSTRACT

System, method, and software of constructing a cohort of sequencing participants. In an embodiment, a system stores sequencing data in electronic format for a plurality of sequencing participants. The system correlates the sequencing data of the sequencing participants with electronic health record data of the sequencing participants received from one or more health care providers. The system provides a cohort graphical user interface to receive cohort criteria from a requestor via a communication network to define a cohort from a population of the sequencing participants, perform electronic data processing on the sequencing data and the electronic health record data for the population of the sequencing participants based on the cohort criteria to identify the cohort of the sequencing participants from the population, and provide the cohort graphical user interface to display cohort information for the cohort to the requestor via the communication network.

### TECHNICAL FIELD

The following disclosure relates to the field of genomics, and more particularly, to the use of genomic data.

### BACKGROUND

Population genomics relies on the detection and exploration of relationships between genetic data and health outcomes in order to power insights that enhance quality of life and reduce mortality. Present solutions for finding insights utilize automated algorithms to scan research datasets describing the genomes of tens or hundreds of thousands of individuals. If a relationship is found between a genetic variant and a health condition, it remains difficult to identify a cohort of individuals having both the genetic variant and the health condition. It also remains difficult to process relevant contextual information for the cohort. Existing processes do not robustly identify this contextual information, nor present it in a manner that is intuitive to interpret.

### SUMMARY

Embodiments described herein associate Electronic Health Record (EHR) data from one or more health care providers/networks with sequencing data stored in a data repository for a population of sequencing participants. Embodiments also provide a graphical user interface (GUI) that allows a user to specify criteria to define a cohort of sequencing participants, and search the EHR data and the sequencing data to identify the sequencing participants of the cohort. The GUI reports out anonymous information for the cohort assembled based on the criteria specified by the user. One technical benefit is the information associated with the cohort includes both EHR data and sequencing data to improve the depth of the available information. Also, the information reported out by the GUI is anonymous to preserve privacy of personal information of the sequencing participants.

In an embodiment, a system comprises a network interface configured to communicate over a communication network, a data repository configured to store sequencing data in electronic format for a plurality of sequencing participants, and a cohort controller comprising a processor and memory. The processor is configured to correlate the sequencing data of the sequencing participants with electronic health record data of the sequencing participants received from one or more health care providers, provide a cohort graphical user interface to receive cohort criteria from a requestor via the communication network to define a cohort from a population of the sequencing participants, perform electronic data processing on the sequencing data and the electronic health record data for the population of the sequencing participants based on the cohort criteria to identify the cohort of the sequencing participants from the population, and provide the cohort graphical user interface to display cohort information for the cohort to the requestor via the communication network.

In an embodiment, a method of constructing a cohort of sequencing participants is disclosed. The method comprises storing, at a data repository, sequencing data in electronic format for a plurality of the sequencing participants. The method further comprises correlating the sequencing data of the sequencing participants with electronic health record data of the sequencing participants received from one or more health care providers, providing a cohort graphical user interface to receive cohort criteria from a requestor via a communication network to define the cohort from a population of the sequencing participants, performing electronic data processing on the sequencing data and the electronic health record data for the population of the sequencing participants based on the cohort criteria to identify the cohort of the sequencing participants from the population, and providing the cohort graphical user interface to display cohort information for the cohort to the requestor via the communication network.

Other embodiments may include computer readable media, other systems, or other methods as described below.

The above summary provides a basic understanding of some aspects of the specification. This summary is not an extensive overview of the specification. It is intended to neither identify key or critical elements of the specification nor delineate any scope particular embodiments of the specification, or any scope of the claims. Its sole purpose is to present some concepts of the specification in a simplified form as a prelude to the more detailed description that is presented later.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a block diagram of a genomic cohort architecture in an illustrative embodiment.

FIG. 2 is a block diagram illustrating generation of sequencing data in an illustrative embodiment.

FIG. 3 is a flow chart illustrating a method of generating sequencing data in an illustrative embodiment.

FIG. 4 is a block diagram of a genomic data system in an illustrative embodiment.

FIG. 5 is a flow chart illustrating a method of constructing or assembling a cohort of sequencing participants in an illustrative embodiment.

FIG. 6 is a flow chart illustrating a method of correlating sequencing data with EHR data in an illustrative embodiment.

FIG. 7 is a block diagram illustrating retrieval of EHR data in an illustrative embodiment.

FIG. 8 is a block diagram illustrating receipt of cohort criteria through a cohort GUI in an illustrative embodiment.

FIG. 9 illustrates a cohort GUI in an illustrative embodiment.

FIG. 10 illustrates a cohort GUI in another illustrative embodiment.

FIG. 11A illustrates identification of a cohort from a population of sequencing participants in an illustrative embodiment.

FIG. 11B illustrates identification of a cohort and a control group from a population of sequencing participants in an illustrative embodiment.

FIG. 12 is a block diagram illustrating a cohort GUI providing cohort information in an illustrative embodiment.

FIG. 13 illustrates a cohort GUI providing cohort information in an illustrative embodiment.

FIG. 14 is a flow chart illustrating a method of generating a list of genetic variants in an illustrative embodiment.

FIG. 15 illustrates a cohort controller providing an anonymized list to a requestor in an illustrative embodiment.

FIG. 16 illustrates a cohort GUI that receives cohort criteria in an illustrative embodiment.

FIG. 17 illustrates a cohort GUI providing cohort information in an illustrative embodiment.

FIG. 18 illustrates a cohort GUI providing cohort information in another illustrative embodiment.

### DETAILED DESCRIPTION

The figures and the following description illustrate specific exemplary embodiments. It will thus be appreciated that those skilled in the art will be able to devise various arrangements that, although not explicitly described or shown herein, embody the principles of the embodiments and are included within the scope of the embodiments. Furthermore, any examples described herein are intended to aid in understanding the principles of the embodiments, and are to be construed as being without limitation to such specifically recited examples and conditions. As a result, the inventive concept(s) is not limited to the specific embodiments or examples described below, but by the claims and their equivalents.

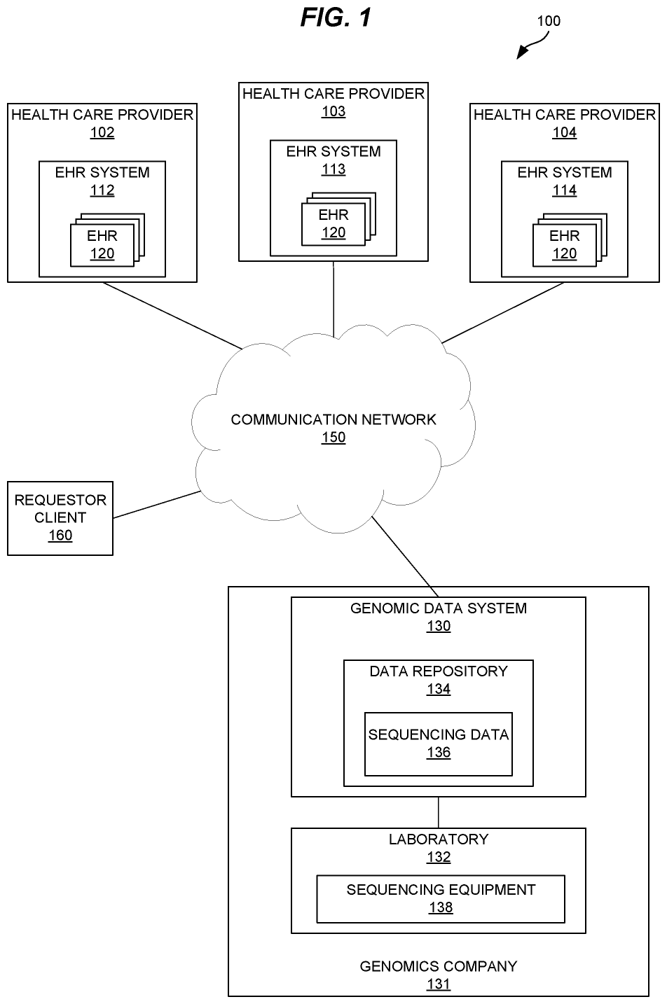

FIG. 1 is a block diagram of a genomic cohort architecture 100 in an illustrative embodiment. At a high level, genomic cohort architecture 100 comprises any combination of systems, components, and/or devices configured to identify a cohort of sequencing participants based on input from a requestor (e.g., health condition(s), genomic criteria, etc.). A sequencing participant comprises an individual that has volunteered or consented to genomic sequencing. In an embodiment, genomic cohort architecture 100 includes one or more EHR systems 112-114 of one or more health care providers 102-104. A health care provider 102-104 is a licensed person or organization that provides healthcare services. A health care provider 102-104 implements an EHR system 112-114 that maintains, tracks, and/or stores EHR data 120 for a plurality of patients. An Electronic Health Record (EHR) is an electronic or digital version of a patient's medical history maintained by a health care provider. EHR data 120 may include patient care information, including demographics (e.g., date of birth, gender, ethnicity, blood type, income, postal code, etc.), progress notes, problems, medications, vital signs, past medical history, immunizations, laboratory data or test results, radiology reports, etc. An EHR system 112-114 may be implemented on a cloud-based or cloud-computing platform, and/or may be implemented on a hardware or server-based platform on-site for the health care provider 102-104.

Genomic cohort architecture 100 further includes a genomic data system 130 of a genomics company 131. In general, a genomics company 131 may perform sample collection, DNA (deoxyribonucleic acid) or genomic sequencing, and secure data storage of the sequencing data generated by the sequencing process. DNA sequencing is a process of exacting a sequence of nucleotides, or bases, in a DNA molecule. Genomic sequencing is a process of determining the entire genetic makeup of an organism. A laboratory 132 associated with the genomics company 131 implements sequencing equipment 138 configured to generate sequencing data 136 from a sample. For example, sequencing equipment 138 may include a DNA sequencer and/or other instruments configured to determine the order of the four bases: G (guanine), C (cytosine), A (adenine) and T (thymine). Genomic data system 130 and/or equipment at laboratory 132 may process the sequencing data 136 to identify variants from the sequencing data 136 (i.e., variant calling). The sequencing data 136 as described herein may therefore include raw DNA or genomic sequences (e.g., order of the bases), and any associated data extracted from the raw sequences, such as variants. Genomic data system 130 is configured to manage and/or process the sequencing data 136, such as by providing secure data storage of the sequencing data 136.

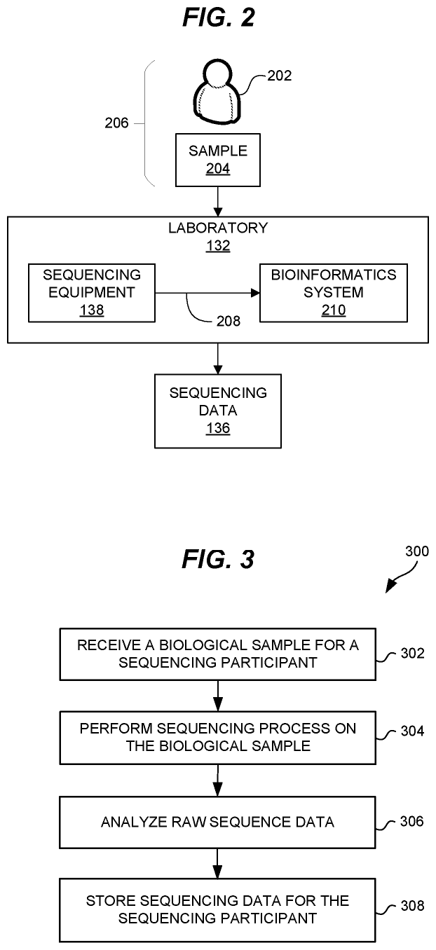

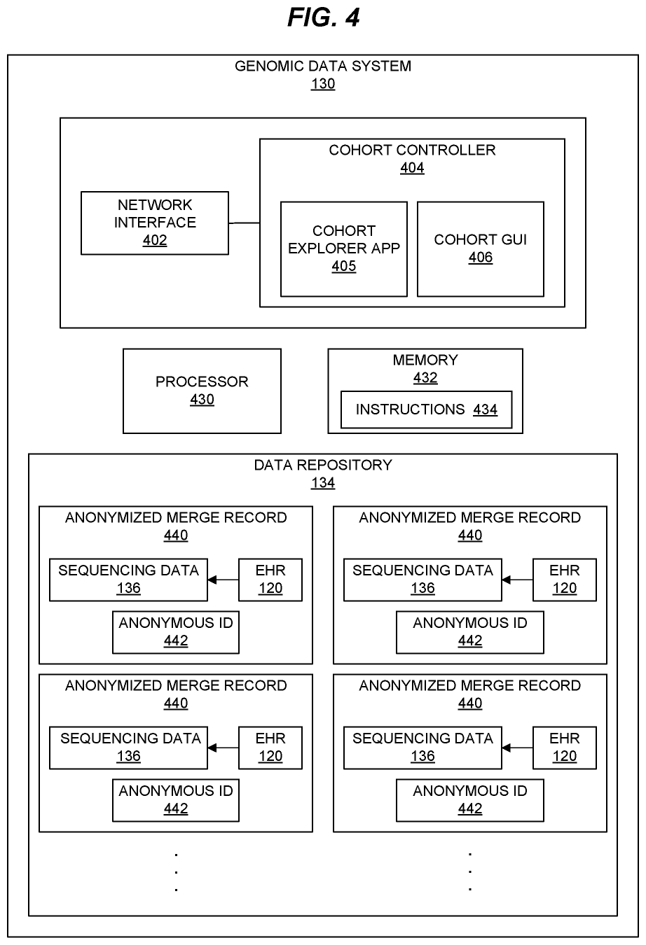

FIG. 2 is a block diagram illustrating generation of sequencing data 136 in an illustrative embodiment. FIG. 3 is a flow chart illustrating a method 300 of generating sequencing data 136 in an illustrative embodiment. The steps of the flow charts described herein are not all inclusive and may include other steps not shown, and the steps may be performed in an alternative order.

A biological sample 204 (e.g., blood, saliva, etc.) of an individual 202 is received at laboratory 132 for sequencing (step 302). An individual 202 that volunteers or consents to genomic sequencing of a biological sample 204 is referred to as a sequencing participant 206. The sequencing equipment 138 at laboratory 132 performs a sequencing process on the biological sample 204 to generate raw sequence data 208 associated with the sequencing participant 206 (step 304). A bioinformatics system 210 may then analyze or otherwise process the raw sequence data 208, such as demultiplexing, alignment, and/or variant calling (step 306). Although bioinformatics system 210 is illustrated as part of laboratory 132 in FIG. 2, bioinformatics system 210 may be implemented outside of or separate from a laboratory 132. Bioinformatics system 210 and/or other systems may further process or analyze the raw sequence data 208, variant information (e.g., research annotation), and/or other information. The result of the above processing is sequencing data 136 for, or associated with, a sequencing participant 206. The sequencing data 136 may comprise data for a whole genome, a subset of the genes that make up a genome, etc. The sequencing data 136 is then stored in secure data storage (step 308), such as in a data repository 134 as shown in FIG. 1.

In FIG. 1, genomic data system 130 is configured to communicate with external systems or devices via a communication network 150. Communication network 150 may comprise a Wide Area Network (WAN), such as the Internet, a telecommunications network, an enterprise network or private network, a Wireless Local Area Network (WLAN), etc., or any combination thereof. As will be described in more detail below, genomic data system 130 is configured to receive or retrieve EHR data 120 associated with sequencing participants 206 as stored in one or more EHR systems 112-114, via communication network 150. Genomic data system 130 is further configured to communicate with a requestor client 160 via communication network 150.

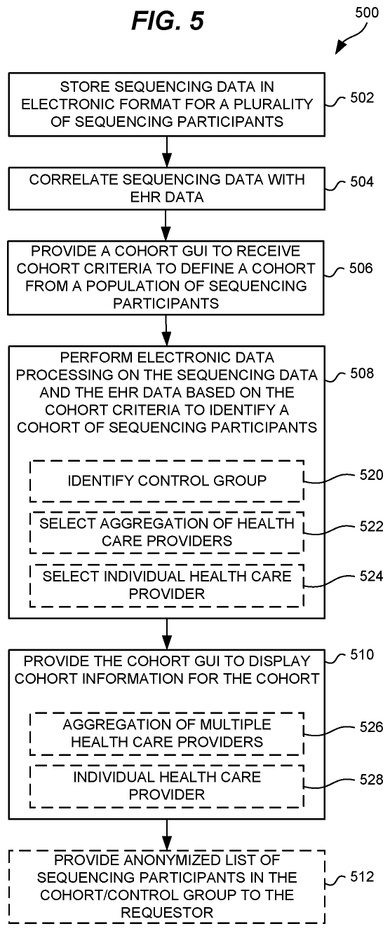

FIG. 4 is a block diagram of genomic data system 130 in an illustrative embodiment. In this embodiment, genomic data system 130 includes the following subsystems: a network interface component 402, a cohort controller 404, and a data repository 134 that operate on one or more platforms. Network interface component 402 may comprise circuitry, logic, hardware, means, etc., configured to exchange messages with external devices or systems. Network interface component 402 may operate using a variety of protocols. Cohort controller 404 may comprise circuitry, logic, hardware, means, etc., configured to assemble a cohort of sequencing participants 206, and report information regarding the cohort. Cohort controller 404 may execute a cohort explorer application 405 to perform the functions or operations described below. Cohort controller 404 also provides a cohort GUI 406, which is a digital interface configured to interact with a user.

Data repository 134 comprises secure data storage configured to store a collection of data. In an embodiment, data repository 134 is configured to store individual records for sequencing participants 206 that are isolated from one another. As shown in FIG. 4, data repository 134 may store anonymized merge records 440 for the sequencing participants 206. An anonymized merge record 440 is a unit of information in electronic format that includes sequencing data 136 for a sequencing participant 206, and also includes EHR data 120 for the sequencing participant 206. Personal information that may indicate a particular person or individual 202 is removed or excluded from the sequencing data 136 and the EHR data 120 so that the anonymized merge record 440 is “anonymized”. The sequencing data 136 and the EHR data 120 for a sequencing participant 206 may be linked (i.e., digitally linked) or merged as the anonymized merge record 440 using an anonymous identifier (ID) 442, such as a digital token, assigned or allocated to the sequencing participant 206. The anonymous ID 442 may be used as an identifier of the sequencing participant 206 in the sequencing data 136 and in the EHR data 120, so the sequencing data 136 and the EHR data 120 of the sequencing participant 206 may be digitally linked via the anonymous ID 442. One technical benefit is the anonymized merge records 440 can be processed, searched, or mined for insights without revealing personal information of sequencing participants 206 to a requestor.

One or more of the subsystems of genomic data system 130 may be implemented on a hardware platform comprised of analog and/or digital circuitry. For example, cohort controller 404 may be implemented on one or more processors 430 that execute instructions 434 (i.e., computer readable code) for software that are loaded into memory 432. A processor 430 comprises an integrated hardware circuit configured to execute instructions 434 to provide the functions of genomic data system 130. Processor 430 may comprise a set of one or more processors or may comprise a multi-processor core, depending on the particular implementation. Memory 432 is a non-transitory computer readable storage medium for data, instructions, applications, etc., and is accessible by processor 430. Memory 432 is a hardware storage device capable of storing information on a temporary basis and/or a permanent basis. Memory 432 may comprise a random-access memory, or any other volatile or non-volatile storage device. One or more of the subsystems of genomic data system 130 may be implemented on a cloud-computing platform or another type of processing platform.

Genomic data system 130 may include various other components not specifically illustrated in FIG. 4.

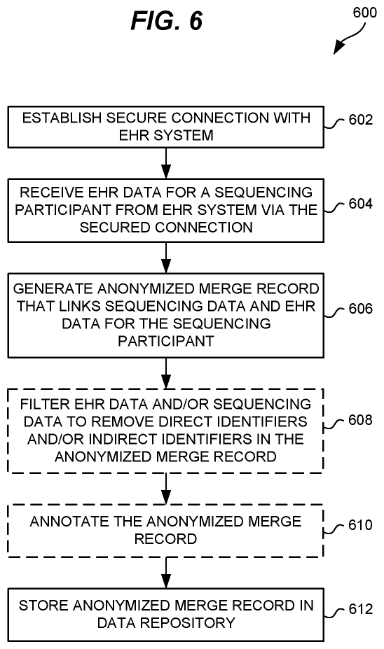

FIG. 5 is a flow chart illustrating a method 500 of constructing or assembling a cohort of sequencing participants 206 in an illustrative embodiment. The steps of method 500 will be described with reference to genomic data system 130 in FIG. 4, but those skilled in the art will appreciate that method 500 may be performed in other systems or devices.

Data repository 134 of genomic data system 130 stores sequencing data 136 in electronic format for a plurality of sequencing participants 206 (step 502). Cohort controller 404 of genomic data system 130 correlates the sequencing data 136 of the sequencing participants 206 with EHR data 120 of the sequencing participants 206 (step 504). For example, cohort controller 404 may retrieve or acquire EHR data 120 (i.e., in electronic format) for one or more of the sequencing participants 206, and link or associate the EHR data 120 of a sequencing participant 206 with the sequencing data 136 stored for the sequencing participant 206 in data repository 134.

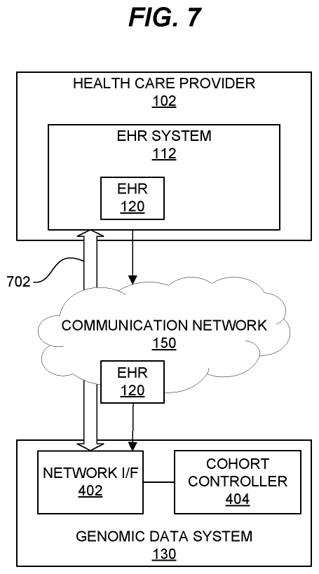

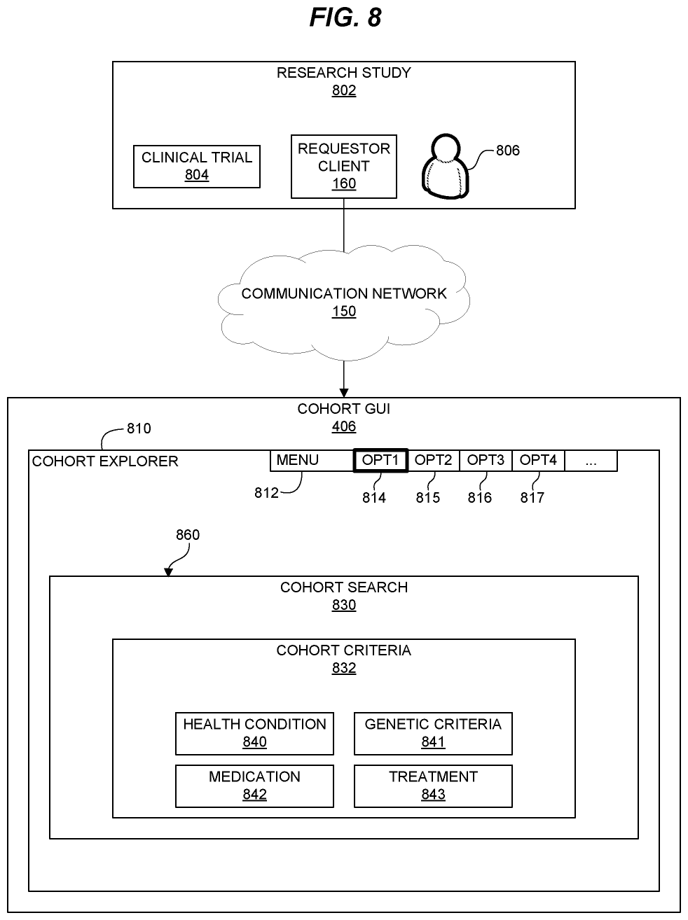

FIG. 6 is a flow chart illustrating a method 600 of correlating sequencing data 136 with EHR data 120 in an illustrative embodiment. To correlate the EHR data 120 with the sequencing data 136 in this embodiment, cohort controller 404 accesses the EHR data 120 from one or more of the EHR systems 112-114 of health care providers 102-104. FIG. 7 is a block diagram illustrating retrieval of EHR data 120 in an illustrative embodiment. In an embodiment, cohort controller 404 may establish a secure connection 702 with an EHR system 112-114 over communication network 150 via network interface component 402 (step 602 of FIG. 6). Cohort controller 404 may then receive the EHR data 120 for one or more sequencing participants 206 from the EHR system(s) 112-114 via the secure connection 702 (step 604 of FIG. 6). In one example, genomics company 131 may receive a biological sample 204 for an individual 202 from a health care provider 102-104 for sequencing at laboratory 132. The EHR system 112 of the health care provider 102-104 may send the EHR data 120 of the individual 202 to genomic data system 130 when sending the biological sample 204 for sequencing (assuming authorizations are provided by the individual 202), or genomic data system 130, such as through cohort controller 404, may retrieve or request the EHR data 120 of the individual 202 from the health care provider 102-104. In FIG. 6, cohort controller 404 generates or creates an anonymized merge record 440 for each of the sequencing participants 206 for which EHR data 120 is received (step 606). As described above, an anonymized merge record 440 includes sequencing data 136 for a sequencing participant 206, and also includes EHR data 120 for the sequencing participant 206. Cohort controller 404 therefore links or merges the sequencing data 136 and the EHR data 120 for the sequencing participants 206 in the anonymized merge record 440. Cohort controller 404 then stores the anonymized merge records 440 in data repository 134 (step 612). One technical benefit is the anonymized merge records 440 include both EHR data and sequencing data to improve the depth of the available information, but privacy of personal information for the sequencing participants 206 is maintained.

As described above, the anonymized merge record 440 is “anonymized” and does not include information that may indicate a particular person or individual 202. In an embodiment, the EHR data 120 received from an EHR system 112-114 may be anonymized. In other words, an EHR system 112-114 may remove any direct identifiers (e.g., Personal Identifiable Information (PII)) of an individual 202 (e.g., patient name, social security number, etc.) from the EHR data 120 before sending to genomic data system 130. Additionally or alternatively, cohort controller 404 may filter the EHR data 120 and/or the sequencing data 136 to remove direct identifiers and/or indirect identifiers from the anonymized merge records 440 (optional step 608). A direct identifier is a unique data attribute related to an individual that allows for univocal correspondence between the data attribute and the individual, such as a name, phone number, date of birth, provider-specific identifier, etc. An indirect identifier is a data attribute not unique to an individual but which contains descriptive information about the individual, such as zip code, county, state, and country of residence, uncommon race or occupation, extreme age, a date or dates of treatment, etc. For example, cohort controller 404 may replace granular addresses and/or dates with broader information. Cohort controller 404 may replace an address with a city or ZIP code, may replace a date with the closest Monday, etc. One technical benefit is cohort controller 404 ensures that privacy of personal information is maintained.

In an embodiment, cohort controller 404 may annotate the anonymized merge record 440 (optional step 610). An annotation may include calculations of risk/severity scores, such as the Madrid Diabetes Risk Score (MADRISC), Diabetes Complications Severity Index DSCI), Estimated Glomerular Filtration Rate (eGFR), Charlson Comorbidity Index (CCI), and others, filling in gaps in information, such as by calculating Body Mass Index (BMI) based on height and weight, etc. One technical benefit of annotation is that cohort controller 404 may improve the information contained in an anonymized merge record 440.

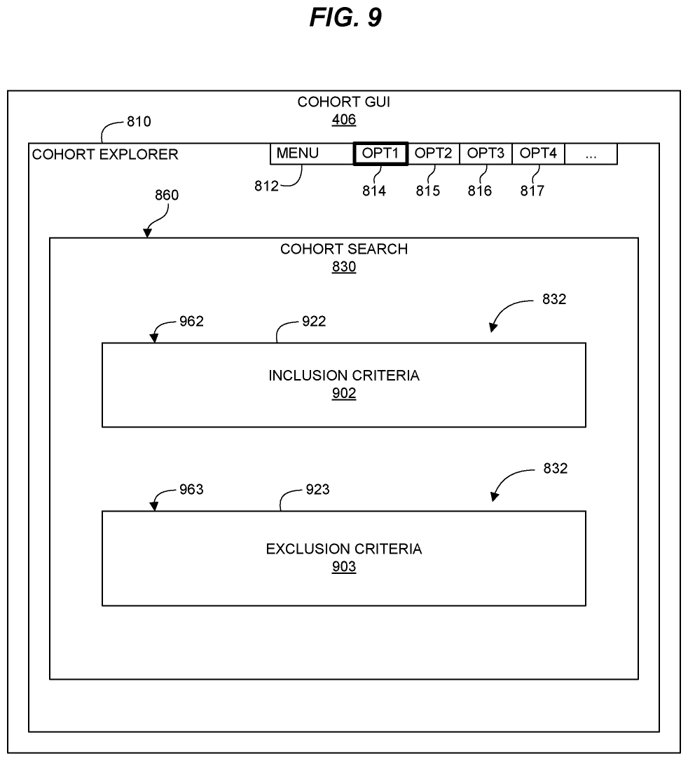

In FIG. 5, cohort controller 404 provides or generates a cohort GUI 406 configured to receive cohort criteria from a requestor via the communication network 150 to define a cohort from a population of sequencing participants 206 (step 506). FIG. 8 is a block diagram illustrating receipt of cohort criteria through cohort GUI 406 in an illustrative embodiment. In this example, cohort GUI 406 provides a cohort explorer interface 810, which is a window, webpage, portal, dashboard, etc., that allows a requestor 806 to interact with cohort controller 404/cohort explorer application 405 via communication network 150. Cohort explorer interface 810 may display a menu 812, which is a graphical element comprising a set of options presented to a requestor 806 to help them find information or execute a function (e.g., menu option 814 (OPT1), menu option 815 (OPT2), menu option 816 (OPT3), menu option 817 (OPT4), etc.). In this example, selection of a menu option (e.g., menu option 814 (OPT1)) may trigger cohort explorer interface 810 to display a cohort search graphical element 830 positioned or located at a display portion 860. A display portion is a section, segment, or part occupying an area or partial area of a GUI. Cohort search graphical element 830 is an interactive graphical element configured to allow a requestor 806 to enter or input cohort criteria 832 to define a cohort of sequencing participants 206. Cohort criteria 832 comprises characteristics, parameters, attributes, or other information to define a cohort from a population of sequencing participants 206. The cohort criteria 832 received at, or input through, cohort search graphical element 830 may include one or more health conditions 840, such as a disease, diagnosis, symptom, etc. The cohort criteria 832 received at, or input through, cohort search graphical element 830 may include genetic criteria 841, such as genes of interest, chromosome position, variant type (deletion, duplication, indel, insertion, single nucleotide, etc.), molecular consequence (frameshift, missense, nonsense, splice site, etc.), P/LP status from ClinVar or a similar source, allele frequency, genetic similarity group (e.g., ancestry) of those with variants, quality scores for the genotypes, etc. The cohort criteria 832 received at, or input through, cohort search graphical element 830 may include one or more medications 842, one or more treatments 843, diagnosis codes (International Classification of Diseases (ICD), Systematized Nomenclature of Medicine Clinical Terms (snomed), etc.), symptoms of interest, lab results, comorbidities, demographics, etc.

In the example in FIG. 8, a requestor 806 may access cohort explorer interface 810 through requestor client 160 over communication network 150 to define a cohort by entering the cohort criteria 832. Although not shown in FIG. 8, requestor 806 may have to set up an account with the provider of the cohort explorer application 405 prior to accessing cohort explorer interface 810, as the cohort explorer application 405 may provide a paid-for or subscription service. Also, requestor 806 may be authenticated by cohort explorer application 405 through a login form/page prior to accessing cohort explorer interface 810. In an embodiment, requestor 806 may define a cohort for a research study 802. One example of a research study 802 is a clinical trial 804, which is a type of research study that tests a medical, surgical, or behavioral intervention in individuals.

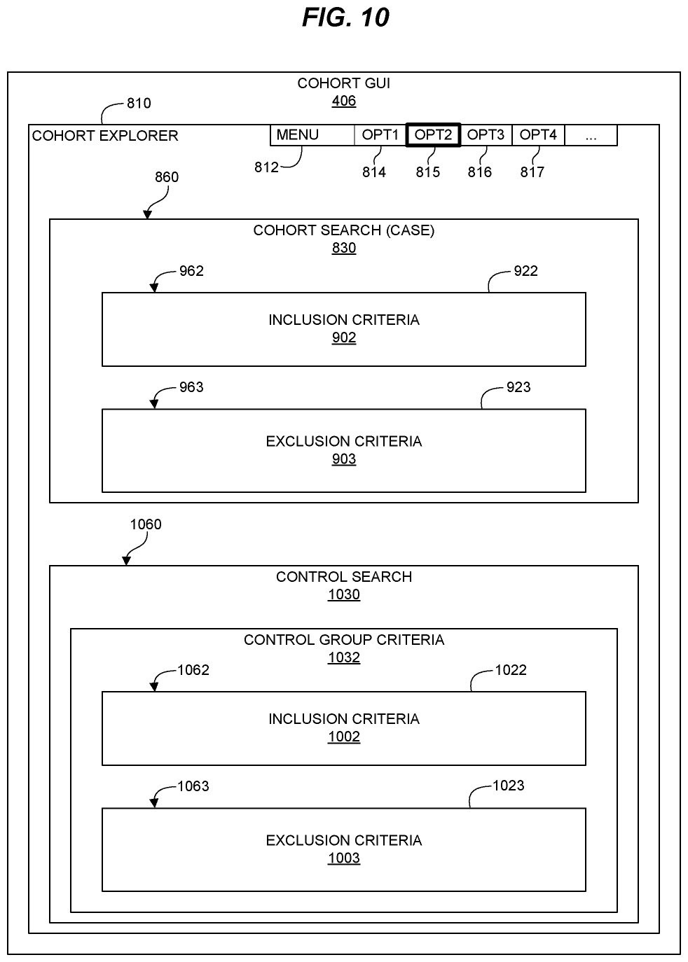

FIG. 9 illustrates a cohort GUI 406 in an illustrative embodiment. The format or configuration of cohort GUI 406 illustrated in FIG. 9 is provided as an example, and other formats or configurations are considered herein. As described above, cohort explorer interface 810 may display one or more graphical control elements that allow a requestor 806 to enter or input the cohort criteria 832, such as for a cohort study. In this embodiment, cohort explorer interface 810 displays an inclusion criteria graphical element 922 positioned or located at a display portion 962 (e.g., first display portion) of cohort search graphical element 830, and configured to receive parameters, attributes, or other inclusion criteria 902 (i.e., of the cohort criteria 832) from a requestor 806 for inclusion in the cohort. Cohort explorer interface 810 may also display an exclusion criteria graphical element 923 positioned or located at a display portion 963 (e.g., a second display portion) of cohort search graphical element 830, and configured to receive parameters, attributes, or other exclusion criteria 903 (i.e., of the cohort criteria 832) from a requestor 806 to be excluded from the cohort. Graphical control elements 922-923 may comprise a list box or drop-down list that enables a requestor 806 to select one or more items, a text box that enables a requestor 806 to input criteria, a combo box that combines a drop-down list or list box and a text box, etc. One technical benefit is a requestor 806 may define what types of sequencing participants 206 to include and what types of sequencing participants 206 to exclude from a cohort based on the inclusion criteria 902 and the exclusion criteria 903.

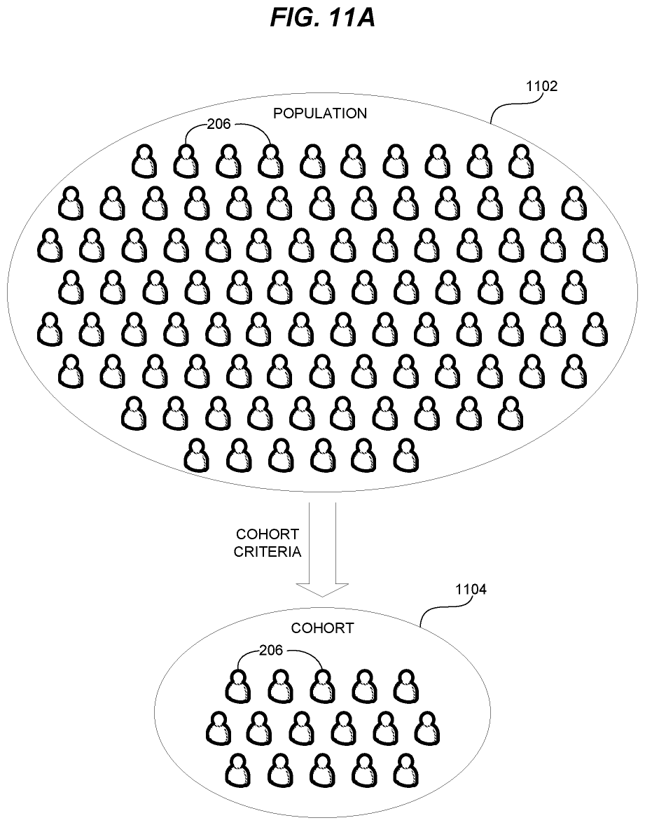

FIG. 10 illustrates a cohort GUI 406 in another illustrative embodiment. The format or configuration of cohort GUI 406 illustrated in FIG. 10 is provided as an example, and other formats or configurations are considered herein. In this embodiment, selection of a menu option (e.g., menu option 815 (OPT2)) may trigger cohort explorer interface 810 to display a cohort search graphical element 830 at a display portion 860 having an inclusion criteria graphical element 922 and an exclusion criteria graphical element 923 as described above. The cohort search graphical element 830 allows a requestor 806 to input the cohort criteria 832 for a cohort (i.e., a case cohort). Selection of the menu option (e.g., menu option 815 (OPT2)) may further trigger cohort explorer interface 810 to display a control search graphical element 1030 positioned or located at a display portion 1060. Control search graphical element 1030 is an interactive graphical element that allows a requestor 806 to enter or input control group criteria 1032 to define a control group of sequencing participants 206, such as for a case-control study. Control group criteria 1032 comprises characteristics, parameters, attributes, or other information to define a control group from a population of sequencing participants 206.

In this example, cohort explorer interface 810 displays an inclusion criteria graphical element 1022 positioned or located at a display portion 1062 (e.g., a first display portion) of control search graphical element 1030, and configured to receive parameters, attributes, or other inclusion criteria 1002 from a requestor 806 for inclusion in the control group. Cohort explorer interface 810 may also display an exclusion criteria graphical element 1023 positioned or located at a display portion 1063 (e.g., a second display portion), and configured to receive parameters, attributes, or other exclusion criteria 1003 from a requestor 806 to be excluded from the control group. One technical benefit is a requestor 806 may define what types of sequencing participants 206 to include and what types of sequencing participants 206 to exclude from a cohort (i.e., case) and a control group (i.e., control).

In FIG. 5, cohort controller 404 performs electronic data processing on the sequencing data 136 and the EHR data 120 for a population of sequencing participants 206 based on the cohort criteria 832 input by the requestor 806 to identify a cohort of sequencing participants 206 from the population (step 508). In other words, cohort controller 404 may parse or search (e.g., automatically in response to the input from the requestor 806) the anonymized merge records 440 for a population of sequencing participants 206 stored in data repository 134 to identify one or more of the anonymized merge records 440 that match the cohort criteria 832 (e.g., an exact match where anonymized merge records 440 contain information exactly matching the cohort criteria 832 and/or a broad match where anonymized merge records 440 contain information related to the cohort criteria 832). Cohort controller 404 may also perform electronic data processing on the sequencing data 136 and the EHR data 120 for the population of sequencing participants 206 based on the control group criteria 1032 input by the requestor 806 to identify a control group of sequencing participants 206 from the population (optional step 520).

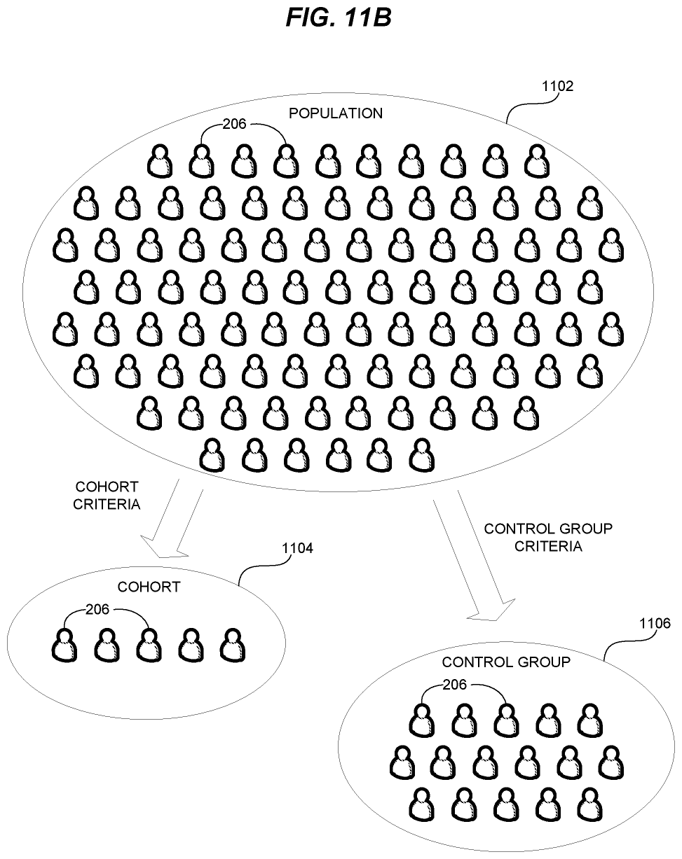

FIG. 11A illustrates identification of a cohort from a population of sequencing participants 206 in an illustrative embodiment. Data repository 134 stores sequencing data 136 and EHR data 120 for a population 1102 of sequencing participants 206. In an embodiment, the population 1102 of sequencing participants 206 may be an aggregation of multiple health care providers 102-104. In other words, sequencing participants 206 in population 1102 may be patients from among multiple health care providers 102-104. Thus, cohort controller 404 may receive a selection from requestor 806, such as through cohort GUI 406, selecting the population 1102 as an aggregation of multiple health care providers 102-104 (see optional step 522 of FIG. 5). In an embodiment, the population 1102 of sequencing participants 206 may be for an individual health care provider 102-104. In other words, sequencing participants 206 in population 1102 may be patients from a single health care provider 102-104. Thus, cohort controller 404 may receive a selection from requestor 806, such as through cohort GUI 406, selecting the population 1102 as an individual health care provider 102-104 (see optional step 524 of FIG. 5). Cohort controller 404 performs electronic data processing on the sequencing data 136 and the EHR data 120 for population 1102 based on the cohort criteria 832 to identify a cohort 1104 of sequencing participants 206 from the population 1102. Cohort 1104 is a group of sequencing participants 206 (i.e., humans) with one or more shared characteristics in the sequencing data 136 and/or the EHR data 120, based on the cohort criteria 832. One technical benefit is cohort explorer interface 810 provides a requestor 806 options to view cohort information 1232 for different populations 1102 (e.g., aggregation or per-health care provider).

Aggregation of data from multiple health care providers 102-104 enables the generation of larger cohorts (or cohorts of sequencing participants 206 with rarer phenotypes or genetic variants) than would be possible via an internal study at a single health care provider. At the same time, because the cohort explorer interface 810 preserves privacy for patients at each health care provider 102-104, it ensures that health care providers 102-104 cannot access PII from sequencing participants 206 at other health care providers. This eliminates the need for complex contractual agreements to be established between all health care providers 102-104 before a requestor 806 can determine whether a cohort of a desired size meets with cohort criteria 832 provided for a study.

FIG. 11B illustrates identification of a cohort and a control group from a population of sequencing participants 206 in an illustrative embodiment. As above, cohort controller 404 performs electronic data processing on the sequencing data 136 and the EHR data 120 for population 1102 based on the cohort criteria 832 to identify a cohort 1104 of sequencing participants 206 from the population 1102. Cohort controller 404 also performs electronic data processing on the sequencing data 136 and the EHR data 120 for population 1102 based on the control group criteria 1032 to identify a control group 1106 of sequencing participants 206 from the population 1102. Control group 1106 is a group of sequencing participants 206 to compare with the cohort 1104, as defined based on the control group criteria 1032.

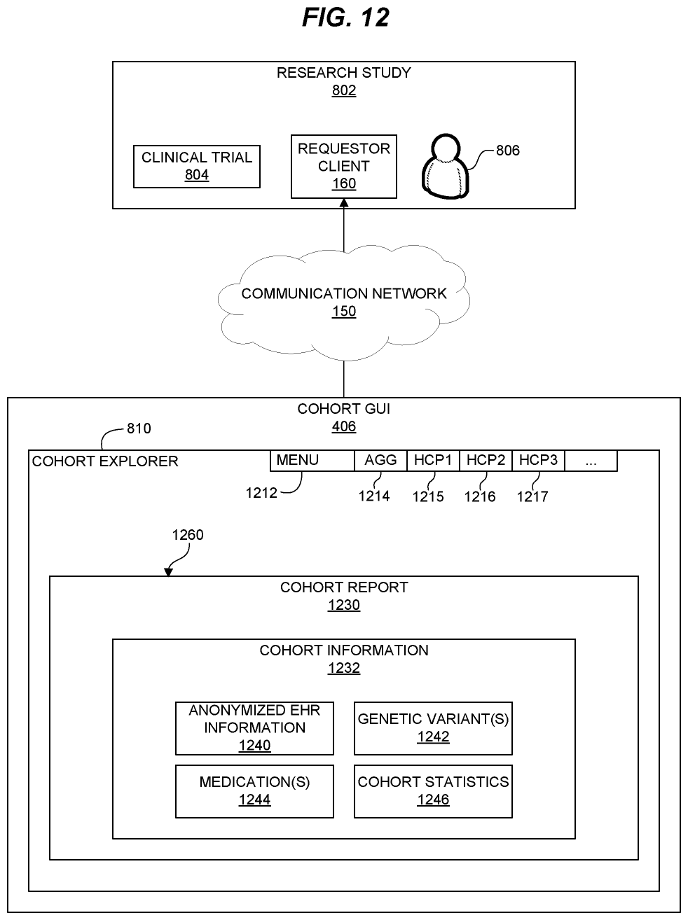

In FIG. 5, cohort controller 404 provides the cohort GUI 406 configured to display cohort information for the cohort 1104 to the requestor 806 via the communication network 150 (step 510). Cohort controller 404 may provide the cohort GUI 406 in real-time or near real-time in response to receiving the cohort criteria 832 from the requestor 806. FIG. 12 is a block diagram illustrating cohort GUI 406 providing cohort information in an illustrative embodiment. In this example, cohort GUI 406 again provides a cohort explorer interface 810, which is a window, webpage, portal, dashboard, etc., that allows a requestor 806 to interact with cohort controller 404/cohort explorer application 405 via communication network 150. Cohort explorer interface 810 may display a cohort report graphical element 1230 positioned or located at a display portion 1260. Cohort report graphical element 1230 is a graphical element that displays or provides cohort information 1232 regarding the cohort 1104. Cohort information 1232 comprises any information about the cohort 1104, the population 1102 from which the cohort 1104 is selected, and/or the sequencing participants 206 of the cohort 1104. For example, the cohort information 1232 may include anonymized EHR information 1240 from the EHR data 120 of the sequencing participants 206 in the cohort 1104, genetic variants 1242 from the sequencing data 136 of the sequencing participants 206 in the cohort 1104, medication information 1244 indicating medications taken or prescribed to the sequencing participants 206 in the cohort 1104, cohort statistics 1246 regarding the cohort 1104 (e.g., number of sequencing participants 206 in the cohort 1104, number of sequencing participants 206 in the population 1102, etc.), and/or other types of information or sequencing results as desired.

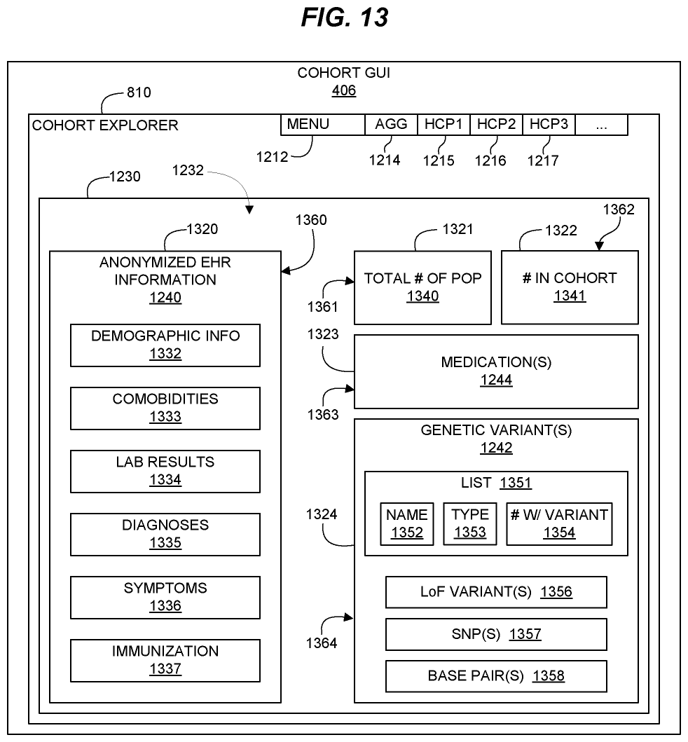

FIG. 13 illustrates a cohort GUI 406 providing cohort information 1232 in an illustrative embodiment. The format or configuration of cohort GUI 406 illustrated in FIG. 13 is provided as an example, and other formats or configurations are considered herein. In an embodiment, cohort explorer interface 810 may display an anonymized EHR graphical element 1320 positioned or located at a display portion 1360 (e.g., first display portion) of the cohort report graphical element 1230, and configured to display anonymized EHR information 1240 of the cohort 1104. The anonymized EHR information 1240 is extracted from the EHR data 120 and is anonymized so as to exclude direct identifiers and/or indirect identifiers regarding the sequencing participants 206 of the cohort 1104. In an embodiment, the anonymized EHR information 1240 may include patient demographic information 1332 (e.g., age, gender, race, etc.), information on comorbidities 1333, lab results 1334, diagnoses 1335, symptoms 1336, immunization information 1337, and/or other information, such as progress notes, vital signs, medical histories, allergies, etc.

To provide cohort statistics 1246, for example, cohort explorer interface 810 may display a statistics graphical element 1321 positioned or located at a display portion 1361 (e.g., second display portion) of the cohort report graphical element 1230, and configured to display a total number 1340 of sequencing participants 206 in the population 1102 under consideration. Cohort explorer interface 810 may display a statistics graphical element 1322 positioned or located at a display portion 1362 (e.g., third display portion) of the cohort report graphical element 1230, and configured to display the number 1341 of sequencing participants 206 in the cohort 1104. To provide medication information 1244, for example, cohort explorer interface 810 may display a medication graphical element 1323 positioned or located at a display portion 1363 (e.g., fourth display portion) of the cohort report graphical element 1230, and configured to display medication information 1244 for sequencing participants 206 in the cohort 1104. It is noted that the medication information 1244 may alternatively or additionally be displayed with the anonymized EHR information 1240 in graphical element 1320.

To display information on genetic variants 1242, for example, cohort explorer interface 810 may display a variant graphical element 1324 positioned or located at a display portion 1364 (e.g., fifth display portion), and configured to display one or more genetic variants 1242 for sequencing participants 206 in the cohort 1104 based on the sequencing data 136. For example, cohort explorer interface 810 may display a list 1351 of genetic variants 1242 found for sequencing participants 206 in the cohort 1104. Each item of the list 1351 may include a name 1352 of a genetic variant 1242, a type 1353 of the genetic variant 1242, and a number 1354 of sequencing participants 206 in the cohort 1104 having the genetic variant 1242, although other information may be included in list 1351. The genetic variants 1242 displayed in graphical element 1324 may be Loss of Function (LoF) variants 1356, Single Nucleotide Polymorphisms (SNPs) 1357, individual base pairs 1358, etc. One technical benefit is cohort explorer interface 810 may display a variety of information regarding the cohort 1104 that may be of interest to the requestor 806 in a clear and visually-pleasing manner.

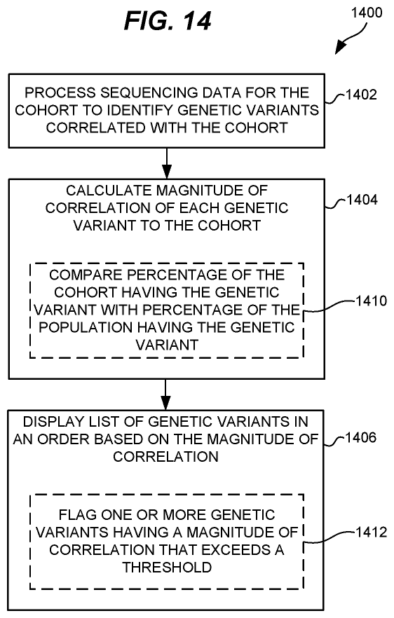

FIG. 14 is a flow chart illustrating a method 1400 of generating a list 1351 of genetic variants 1242 in an illustrative embodiment. Cohort controller 404 processes the sequencing data 136 for the cohort 1104 to identify genetic variants 1242 correlated with the cohort 1104 (step 1402). In other words, cohort controller 404 searches for genetic variants 1242 indicated in the sequencing data 136 of the sequencing participants 206 in the cohort 1104. Cohort controller 404 then calculates or determines a magnitude of correlation for each of the genetic variants 1242 to the cohort 1104 (step 1404). In an embodiment, cohort controller 404 may calculate the magnitude of correlation of a genetic variant 1242 by comparing the fraction or percentage (i.e., first percentage) of the cohort 1104 having the genetic variant 1242 with the fraction or percentage (i.e., second percentage) of the population 1102 having the genetic variant 1242 (optional step 1410). For example, the difference between the first percentage and the second percentage, a ratio of the first percentage to the second percentage, and/or another type of comparison indicates the magnitude of correlation. Cohort controller 404 then provides the cohort GUI 406 to display the list 1351 of genetic variants 1242 in an order based on the magnitude of correlation (step 1406). For example, cohort GUI 406 may display the genetic variants 1242 (i.e., in variant graphical element 1324) in an order of higher magnitude of correlation to lower magnitude of correlation. Cohort GUI 406 may also flag one or more genetic variants 1242 having a magnitude of correlation that exceeds a threshold, such as for further investigation or study (optional step 1412). One technical benefit is the genetic variants 1242 having the strongest correlation to the cohort 1104 are clearly displayed through cohort explorer interface 810.

As described above, a cohort 1104 as described herein may be an aggregation of a plurality of health care providers 102-104, or may be limited to an individual health care provider 102-104. Thus, cohort explorer interface 810 may display the cohort information 1232 for an aggregation of multiple health care providers 102-104 (see optional step 526 of FIG. 5), and/or display the cohort information 1232 for individual health care providers 102-104 or per-health care provider 102-104 (see optional step 528 of FIG. 5). In an embodiment as in FIG. 13, cohort explorer interface 810 may display a menu 1212, which is a graphical element comprising a set of options (e.g., menu option 1214 (AGG), menu option 1215 (HCP1), menu option 1216 (HCP2), menu option 1217 (HCP3), etc.). Selection of a menu option (e.g., menu option 1214 (AGG)) may trigger cohort explorer interface 810 to display cohort information 1232 for an aggregation of multiple health care providers 102-104. Thus, the population 1102 of sequencing participants 206, from which the cohort 1104 is selected, is across multiple health care providers 102-104. Selection of another menu option may trigger cohort explorer interface 810 to display cohort information 1232 for an individual health care provider 102-104. For example, selection of menu option 1215 (HCP1) may trigger cohort explorer interface 810 to display cohort information for health care provider 102. Thus, the population 1102 of sequencing participants 206, from which the cohort 1104 is selected, is limited to patients of health care provider 102. Selection of menu option 1216 (HCP2) may trigger cohort explorer interface 810 to display cohort information 1232 for health care provider 103. Thus, the population 1102 of sequencing participants 206, from which the cohort 1104 is selected, is limited to patients of health care provider 103. Selection of menu option 1217 (HCP3) may trigger cohort explorer interface 810 to display cohort information 1232 for health care provider 104. Thus, the population 1102 of sequencing participants 206, from which the cohort 1104 is selected, is limited to patients of health care provider 104.

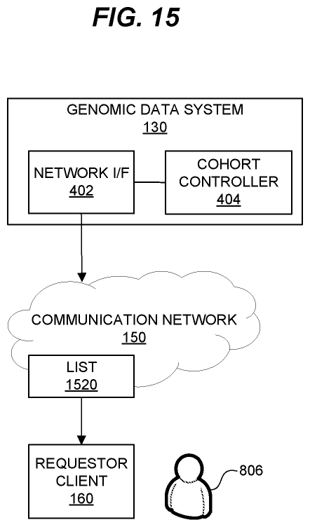

In FIG. 5, cohort controller 404 may provide an anonymized list of the sequencing participants 206 in the cohort 1104 to the requestor 806 via the communication network 150 (optional step 512). FIG. 15 illustrates cohort controller 404 providing an anonymized list to the requestor 806 in an illustrative embodiment. Cohort controller 404 (through network interface component 402) may send the anonymized list 1520 to requestor client 160 via communication network 150, such as through email, via secure file transfer protocol, or another method. The anonymized list 1520 includes (anonymous) information regarding sequencing participants 206 in the cohort 1104. In an embodiment, the anonymized list 1520 may further include (anonymous) information regarding sequencing participants 206 in the control group 1106. One technical benefit is the requestor 806 may use the anonymized list 1520 as a starting point in assembling a group for a research study, or for further study, with private information of the sequencing participants 206 protected. Additionally, cohort controller 404 may allow for the export of the cohort-defining query (software code) that may be used to re-create the cohort 1104 outside of genomic data system 130. Also, cohort controller 404 may allow for the ability to save the definition for generating the cohort 1104 and/or anonymized list 1520 so that the requestor 806 can return to the definition and make edits if desired.

### Example

In the following example, additional processes, systems, and methods may be described in the context of defining a cohort 1104. The processes, systems, and methods described in this example may be incorporated in embodiments described above as desired.

Biological samples 204 for a plurality of individuals 202 are received at laboratory 132 for sequencing. Laboratory procedures related to genetics may include accessioning, sample plating, storage, extraction, library preparation, enrichment, and sequencing processes. These processes acquire genetic material from a sample, separate the genetic material from other constituents, duplicate the genetic material, and quantify the genetic material order to determine a swathe of sequence data, such as an exome or entire genome for a subject (e.g., a human, an animal, a pathogen, an organelle, etc.). Thus, the sequencing equipment 138 at laboratory 132 performs a sequencing process on the biological samples 204 to generate raw sequence data for the individuals.

Sequencing may be performed according to any of a variety of techniques, including short-read and long-read techniques. In one embodiment, the sequencing is performed as Sequencing by Synthesis (SBS) at genetic analyzer equipment. For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers. In one embodiment, the chemical identifiers comprise nucleotide sequences that are detectable during the sequencing process to determine a corresponding Laboratory Sample Identifier (LSI).

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

Sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

In some embodiments, genetic material is used for detection of a pathogen rather than for sequencing. Detecting a pathogen may involve the use of a real-time Polymerase Chain Reaction (PCR) system that performs PCR. The real-time PCR system may further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen may thereby forego sequencing in some embodiments.

Raw sequence data generated during synthesis is stored in a file format, such as Binary Base Call (BCL). This raw data may be fed to an analytical pipeline, such as a cloud-based computing environment. Raw sequence data may be processed by the pipeline into a second format, such as a text-based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics, such as quality indicator metrics.

The called sequence data may be provided to a data analyst via a User Interface (UI), such as a GUI presented via a display. The technician may then validate the resulting called sequence data and release it for reporting to subjects, health care providers, and/or scientists. The raw sequence data, the called sequence data, and/or any annotations provided by a data analyst forms the sequencing data 136 that is stored in data repository 134.

The individuals 202, for which sequencing data 136 is generated, forms a population 1102 of sequencing participants 206. A genomic data system 130 as described herein associates EHR data 120 with the sequencing data 136 stored in data repository 134. Thus, the EHR data 120 and sequencing data 136 may be mined for the population 1102 of sequencing participants 206 using the cohort explorer interface 810.

Cohort controller 404 of genomic data system 130 receives EHR data 120 for the sequencing participants 206 from EHR systems 112-114 of one or more health care providers 102-104. Direct identifiers (e.g., patient names, social security numbers, etc.) are removed from the EHR data 120 as submitted to genomic data system 130. The EHR data 120 may be formatted in a standard Observational Medical Outcomes Partnership (OMOP) format (e.g., v5.3, v5.4, etc.), and/or a local EHR format, and sent over communication network 150 via secure file transfer protocol. Cohort controller 404 may transform the EHR data 120 from each health care providers 102-104 into a uniform OMOP version (e.g., OMOP v5.4), and make a copy of the EHR data 120. Cohort controller 404 may review the EHR data 120 to remove any direct identifiers (if included) and/or in-direct identifiers that remain in the EHR data 120. Cohort controller 404 may additionally perform various annotations upon the EHR data 120. Cohort controller 404 then correlates the sequencing data 136 of each sequencing participant 206 with the EHR data 120 of the sequencing participant 206 (e.g., generate anonymized merge record 440 for the sequencing participant 206 that includes sequencing data 136 and EHR data 120).

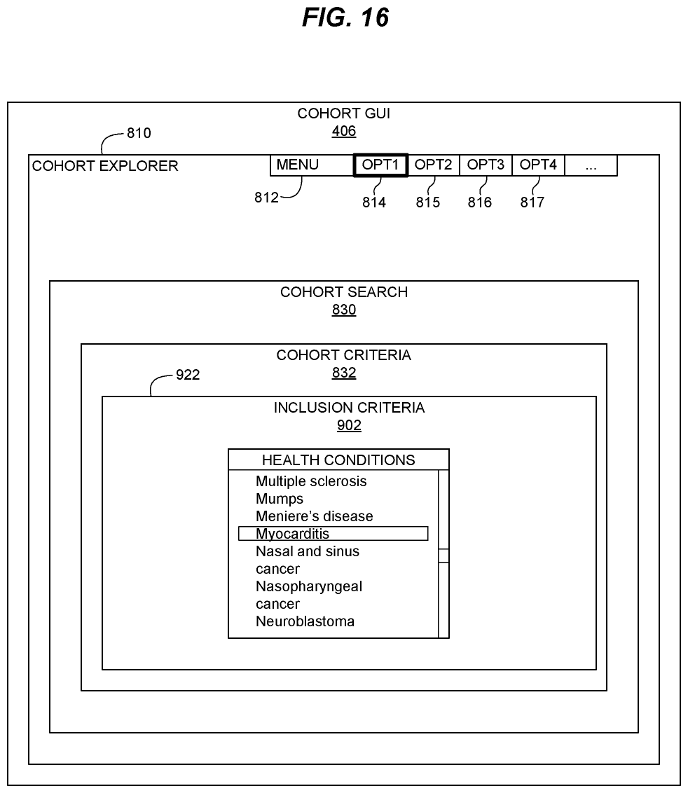

Cohort controller 404 provides cohort GUI 406 (i.e., cohort explorer interface 810) that enables a requestor 806 to define a cohort 1104, such as for a research study 802. FIG. 16 illustrates a cohort GUI 406 that receives cohort criteria 832 in an illustrative embodiment. In this example, cohort GUI 406 provides cohort explorer interface 810 that allows a requestor 806 to interact with cohort controller 404/cohort explorer application 405 via communication network 150 (see also, FIG. 8). Cohort explorer interface 810 displays a cohort search graphical element 830 that allows a requestor 806 to enter or input cohort criteria 832 to define a cohort 1104 of sequencing participants 206. For example, the research study 802 may be for a particular health condition 840 being investigated. In FIG. 16, cohort explorer interface 810 displays an inclusion criteria graphical element 922 configured to receive inclusion criteria 902 from the requestor 806 for inclusion in the cohort 1104. In this example, inclusion criteria graphical element 922 comprises a drop-down list of health conditions 840 for inclusion in the cohort 1104, and the health condition 840 of “Myocarditis” is selected by the requestor 806. In this example, each health condition 840 is assigned a predefined list of one or more Current Procedural Terminology (CPT) codes, International Classification of Diseases (ICD) codes, snomed codes, or the like that are associated with the health condition 840 (i.e., that refer to treatment for, or diagnosis of, the health condition). This enables cohort controller 404 to identify sequencing participants 206 for inclusion in the cohort 1104 based on CPT, ICD, or snomed codes in EHR data 120 for the sequencing participants 206. However, it is understood that the health condition 840 being investigated may be entered into inclusion criteria graphical element 922 in other ways, such as by directly entering CPT codes, ICD codes, snomed codes, or the like.

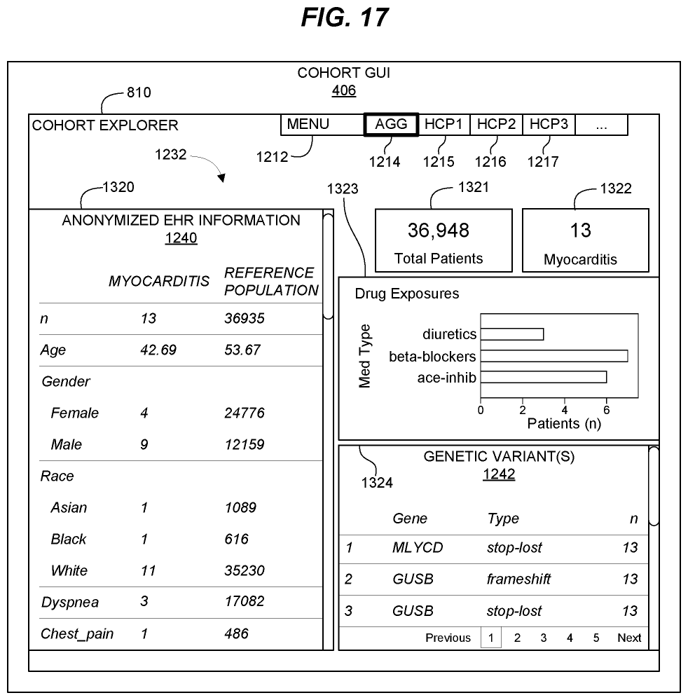

Cohort controller 404 performs electronic data processing on the sequencing data 136 and the EHR data 120 for the population 1102 of sequencing participants 206 based on the cohort criteria 832 (i.e., health condition=Myocarditis) to identify a cohort 1104 of sequencing participants 206 from the population 1102. In other words, cohort controller 404 processes the EHR data 120 for the population 1102 of sequencing participants 206 to identify sequencing participants 206 that have the health condition 840 of “Myocarditis”, which defines the cohort 1104. Cohort controller 404 then provides the cohort GUI 406 to display cohort information 1232 for the cohort 1104. FIG. 17 illustrates a cohort GUI 406 providing cohort information 1232 in an illustrative embodiment. In this example, cohort GUI 406 again provides a cohort explorer interface 810 that displays or provides cohort information 1232 regarding the cohort 1104 related to Myocarditis. Cohort explorer interface 810 displays a graphical element 1320 configured to display anonymized EHR information 1240 of the cohort 1104. The anonymized EHR information 1240 may include patient demographic information 1332 (e.g., age, gender, race, etc.), information on comorbidities 1333, lab results 1334, diagnoses 1335, symptoms 1336 (e.g., dyspnea, chest pain, etc.), immunization information 1337, and/or other information. To provide cohort statistics 1246, for example, cohort explorer interface 810 displays a graphical element 1321 configured to display a total number 1340 of sequencing participants 206 in the population 1102 under consideration (e.g., “36,948”), and a graphical element 1322 configured to display the number 1341 of sequencing participants 206 in the cohort 1104 (e.g., “13”). To provide medication information 1244, cohort explorer interface 810 displays a graphical element 1323 configured to display medication information 1244 for sequencing participants 206 in the cohort 1104 (i.e., “drug exposures”).

To display information on genetic variants 1242, cohort explorer interface 810 displays a graphical element 1324 configured to display a list of genetic variants 1242 for sequencing participants 206 in the cohort 1104. Cohort controller 404 identifies genetic variants 1242 within the cohort 1104 that are strongly correlated with the health condition 840 being studied. Cohort explorer interface 810 lists these genetic variants 1242 in order of the magnitude of correlation with the health condition 840 being investigated.

Cohort controller 404 may also identify related health conditions (i.e., comorbidities 1333) that are strongly correlated with the health condition 840 being investigated, in a similar manner described above for related genetic variants 1242. The related health conditions may comprise a predefined list of health conditions. This data may provide additional insights into further avenues of study.

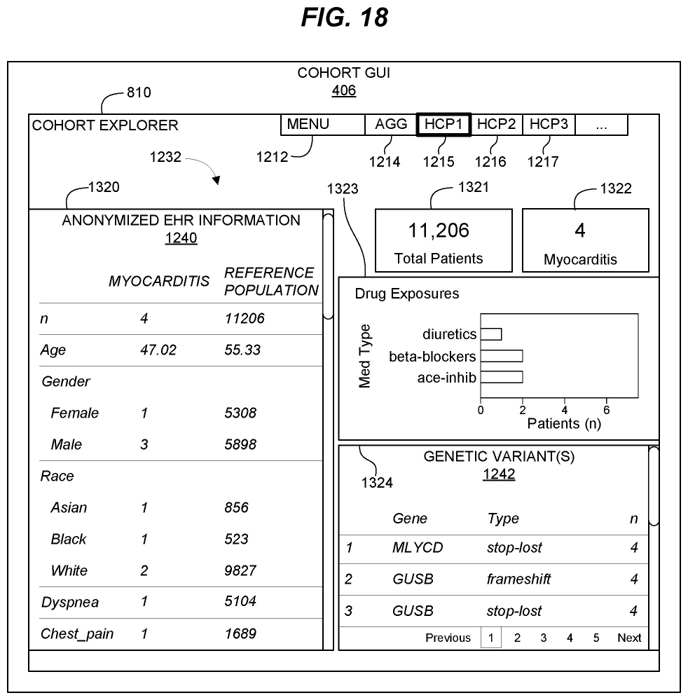

In FIG. 17, the menu option (e.g., menu option 1214 (AGG)) is selected to display cohort information 1232 for an aggregation of multiple health care providers 102-104. Thus, the population 1102 of sequencing participants 206, from which the cohort 1104 is selected, is across multiple health care providers 102-104. FIG. 18 illustrates a cohort GUI 406 providing cohort information 1232 in another illustrative embodiment. In FIG. 18, the menu option (e.g., menu option 1215 (HCP1)) is selected to display cohort information 1232 for an individual health care provider 102. Thus, cohort explorer interface 810 may display cohort information 1232 for an aggregation of multiple health care providers 102-104, or per-health care provider 102-104.

Systems and methods discussed above provide a technical benefit by preserving the privacy of populations 1102 whose sequencing data 136 is being researched, while also enabling members of the population 1102 to opt in to research studies 802, such as clinical trials 804. This may help to facilitate, for example, tailored dosages for pharmaceuticals based on genetic variants 1242, identification of adverse combinations of pharmaceuticals and genetic variants 1242, and/or phenotypic behaviors that result in notably increased risk for those who exhibit certain genetic variants 1242.

Although specific embodiments were described herein, the scope of the invention is not limited to those specific embodiments. The scope of the invention is defined by the following claims and any equivalents thereof.

What is claimed is:

1. A system, comprising:
a network interface configured to communicate over a communication network;
a data repository configured to store sequencing data in electronic format for a plurality of sequencing participants; and
a cohort controller comprising a processor and memory, the processor configured to:
correlate the sequencing data of the sequencing participants with electronic health record data of the sequencing participants received from one or more health care providers;
provide a cohort graphical user interface to receive cohort criteria from a requestor via the communication network to define a cohort from a population of the sequencing participants;
perform electronic data processing on the sequencing data and the electronic health record data for the population of the sequencing participants based on the cohort criteria to identify the cohort of the sequencing participants from the population; and
provide the cohort graphical user interface to display cohort information for the cohort to the requestor via the communication network.

2. The system of claim 1, wherein the processor is further configured to:
provide an anonymized list of the sequencing participants in the cohort to the requestor via the communication network.

3. The system of claim 1, wherein the processor is further configured to:
receive a selection from the requestor selecting the population as an aggregation of multiple ones of the health care providers.

4. The system of claim 1, wherein the processor is further configured to:
receive a selection from the requestor selecting the population as an individual one of the health care providers.

5. The system of claim 1, wherein the processor is further configured to:
receive the electronic health record data for one or more of the sequencing participants via a secure connection;
generate an anonymized merge record for each of the sequencing participants for which the electronic health record data is received, wherein the anonymized merge record of a sequencing participant includes the sequencing data and the electronic health record data for the sequencing participant; and
store anonymized merge records in the data repository.

6. The system of claim 5, wherein the processor is further configured to:
filter at least one of the electronic health record data and the sequencing data to remove direct identifiers and/or indirect identifiers from the anonymized merge records.

7. The system of claim 1, wherein the processor is further configured to:
provide the cohort graphical user interface to display a cohort search graphical element configured to allow the requestor to input the cohort criteria.

8. The system of claim 7, wherein the processor is further configured to:
provide the cohort graphical user interface to display an inclusion criteria graphical element positioned at a first display portion of the cohort search graphical element, and configured to receive inclusion criteria from the requestor for inclusion in the cohort.

9. The system of claim 8, wherein the processor is further configured to:
provide the cohort graphical user interface to display an exclusion criteria graphical element positioned at a second display portion of the cohort search graphical element, and configured to receive exclusion criteria from the requestor to be excluded from the cohort.

10. The system of claim 1, wherein the processor is further configured to:
provide the cohort graphical user interface to display a cohort report graphical element configured to display the cohort information.

11. The system of claim 10, wherein:
the cohort information includes at least one of anonymized electronic health record information from the electronic health record data of the sequencing participants in the cohort, one or more genetic variants from the sequencing data of the sequencing participants in the cohort, medications prescribed to the sequencing participants in the cohort, and statistics regarding the cohort.

12. The system of claim 10, wherein the processor is further configured to:
provide the cohort graphical user interface to display an anonymized electronic health record graphical element positioned at a display portion of the cohort report graphical element, and configured to display anonymized electronic health record information from the electronic health record data of the sequencing participants in the cohort.

13. The system of claim 10, wherein the processor is further configured to:
provide the cohort graphical user interface to display a statistics graphical element positioned at a display portion of the cohort report graphical element, and configured to display a total number of the sequencing participants in the population.

14. The system of claim 10, wherein the processor is further configured to:
provide the cohort graphical user interface to display a statistics graphical element positioned at a display portion of the cohort report graphical element, and configured to display a number of the sequencing participants in the cohort.

15. The system of claim 10, wherein the processor is further configured to:
provide the cohort graphical user interface to display a variant graphical element positioned at a display portion of the cohort report graphical element, and configured to display one or more genetic variants for the sequencing participants in the cohort based on the sequencing data.

16. The system of claim 15, wherein the processor is further configured to:
process the sequencing data for the cohort to identify the genetic variants correlated with the cohort;
calculate a magnitude of correlation for each of the genetic variants to the cohort; and
provide the cohort graphical user interface to display the variant graphical element configured to display a list of the genetic variants in an order based on the magnitude of correlation.

17. The system of claim 16, wherein the processor is further configured to:
calculate the magnitude of correlation of a genetic variant by comparing a first percentage of the cohort having the genetic variant with a second percentage of the population having the genetic variant.

18. A method of constructing a cohort of sequencing participants, the method comprising:
storing, at a data repository, sequencing data in electronic format for a plurality of the sequencing participants;
correlating the sequencing data of the sequencing participants with electronic health record data of the sequencing participants received from one or more health care providers;
providing a cohort graphical user interface to receive cohort criteria from a requestor via a communication network to define the cohort from a population of the sequencing participants;
performing electronic data processing on the sequencing data and the electronic health record data for the population of the sequencing participants based on the cohort criteria to identify the cohort of the sequencing participants from the population; and
providing the cohort graphical user interface to display cohort information for the cohort to the requestor via the communication network.

19. The method of claim 18, further comprising:
providing an anonymized list of the sequencing participants in the cohort to the requestor via the communication network.

20. A non-transitory computer readable medium embodying programmed instructions executed by a processor, wherein the instructions direct the processor to implement a method of constructing a cohort of sequencing participants, the method comprising:
storing, at a data repository, sequencing data in electronic format for a plurality of the sequencing participants;
correlating the sequencing data of the sequencing participants with electronic health record data of the sequencing participants received from one or more health care providers;
providing a cohort graphical user interface to receive cohort criteria from a requestor via a communication network to define the cohort from a population of the sequencing participants;
performing electronic data processing on the sequencing data and the electronic health record data for the population of the sequencing participants based on the cohort criteria to identify the cohort of the sequencing participants from the population; and
providing the cohort graphical user interface to display cohort information for the cohort to the requestor via the communication network.
